(19) United States
(12) Patent Application Publication
(10) Pub. No.: US 2013/0204392 A1
(43) Pub. Date: Aug. 8, 2013

(54) METHOD FOR PROMOTING STEM CELL CHONDROGENESIS

(71) Applicants: AGENCY FOR SCIENCE, TECHNOLOGY AND RESEARCH, Singapore (SG); NATIONAL UNIVERSITY OF SINGAPORE, Singapore (SG); NATIONAL UNIVERSITY HEALTH SYSTEM, Singapore (SG)

(72) Inventors: Bee Khuan Jaslyn Law, Singapore (SG); Ai Yu He, Singapore (SG); Hong Yee Low, Singapore (SG); Yingnan Wu, Singapore (SG); Zheng Yang, Singapore (SG); Eng Hin Lee, Singapore (SG)

(73) Assignees: AGENCY FOR SCIENCE, TECHNOLOGY AND RESEARCH, Singapore (SG); NATIONAL UNIVERSITY HEALTH SYSTEM, Singapore (SG); NATIONAL UNIVERSITY OF SINGAPORE, Singapore (SG)

(21) Appl. No.: 13/755,529

(22) Filed: Jan. 31, 2013

(30) Foreign Application Priority Data
Jan. 31, 2012 (SG) 201200694-6

(51) Int. Cl. A61K 35/32; C12N 5/071

(52) U.S. Cl. CPC A61K 35/32; C12N 5/0655
USPC 623/23.72; 435/377

(57) ABSTRACT

There is provided a method for promoting stem cell chondrogenesis, comprising the step of culturing a population of stem cells on a plurality of imprints disposed on a substrate, the imprints being configured to selectively promote chondrogenesis of the stem cells.

### TECHNICAL FIELD

The present invention generally relates to a method for promoting stem cell chondrogenesis. The present invention also relates to a cartilage graft.

### BACKGROUND

Research efforts have been focused on the development of tissue engineering as an alternative to drug therapy, gene therapy or even whole organ transplant. In particular, one area of tissue engineering focuses on the development of artificial matrices, i.e., the creation of artificial scaffolds to support cells as they grow. These artificial supports have become more complex with technological developments, transforming them from simple polymer films to fibrous matrices with decreasing fiber-size-scale and designed to closely resemble the natural extracellular matrix (ECM), which supports tissue growth and repair.

In such engineering methods, the tissues used are usually comprised of an assembly of cells entwined or supported by the ECM. In particular, the types of tissue found in vertebrates may include nerve, muscle, epithelial, and connective tissues etc. Although these tissues, if damaged or impaired, may be restored via ex-vivo methods, such treatment methods of defective tissues still remain a significant clinical challenge. To illustrate further, some of these tissues may possess poor self-regenerative capacity due to low cellular mitotic activities, low supply of progenitor cells and its avascularization. Other ex-vivo methods explored to overcome the above issues, such as using culture-expanded autologous cells for defective tissue treatment often do not provide sufficient cells for repair. The initial cells used for culturing may also be difficult to extract. Furthermore, ex-vivo expansion methods may also result in the loss of the cells' functional phenotype which is undesirable.

An alternative approach to defective tissue repair may be in-vitro tissue engineering. In-vitro systems integrated with the appropriate use of isolated cells, cell substitutes to replace those damaged cells or tissue-inducing substances, such as growth and differentiation factors, supported on scaffolds may overcome the limitations mentioned above.

It is to be noted that the use of isolated cells, cell substitutes or tissue-inducing substances alone can only be used when the tissue defects are small and well contained. The in-vitro approach, i.e., growing cells isolated from their original biological surroundings on supporting scaffolds, has become increasingly popular. In such an integrated approach, the types of ECM used for coating the scaffold, together with the selection of cells and the topography of the scaffold can play a pivotal role in guiding cells to grow, synthesize extracellular matrix and other biological molecules as well as facilitating the formation of functional tissues. On top of that, the ECM used for coating may determine how the cells interact within the micro-environment by allowing specific cell-cell or cell-matrix interactions to occur. In addition, the right selection of cell types for use can lead to ease of extraction, high proliferation capacity along with the ability of further cell differentiation. In particular, such cells may also respond to the topographical cues of the scaffold support that subsequently influences cell proliferation and differentiation. Lastly, the topography of the scaffold support may also affect the cell responses.

With regard to the last factor, some important scaffold design principles that have been widely accepted may include high surface area, biodegradability and biocompatibility of the material, mechanical integrity to maintain the predesigned tissue structure and enable positive interactions with cells (enhanced cell adhesion, growth, migration, and differentiated function). Several known scaffold fabrication methods, such as phase separation, electro-spinning, micro-topographical effects on micro-beads prepared by gel formation have been proposed to improve the positive interactions. Yet, the problem of random topography achieved and the limited degree of spatial control still persists. Consequently, variation within the fibrous structure of the scaffold makes it difficult to investigate the response of the cells to random surface topography.

Accordingly, there is a need to develop a topographical scaffold for tissue engineering that integrates the selection of cell types and ECM used for coating the nano-topographical scaffold so as to mitigate the limitations discussed above.

There is a need to develop a method that allows for a selective activation and differentiation of cells on various topological surfaces.

### SUMMARY

According to a first aspect, there is provided a method for promoting stem cell chondrogenesis, comprising the step of culturing a population of stem cells on a plurality of imprints disposed on a substrate, said imprints being configured to selectively promote chondrogenesis of the stem cells.

Advantageously, by selecting the type of imprints on the substrate, the activation and differentiation of stem cells under chondrogenic conditions may be selectively controlled. Depending on the type of imprints on the substrate surface, the chondrogenesis of the stem cells may be increased as compared to that of identical stem cells on a comparative substrate not having any imprints thereon. The chondrogenesis of the stem cells may also be increased as compared to a surface that has random nanotopography (that is, a surface in which the nanotopography cannot be predicted or formed according to a desired or predictive pattern).

Advantageously, spatially-controlled nano-imprints on a substrate film can be used as a scaffold and coated with an extra-cellular matrix of chondroitin sulfate (CS) for enhancement or promotion of in-vitro stem cell chondrogenesis for effective cartilage tissue engineering. Specifically, certain types of nano-imprints enhanced stem cells chondrogenesis while another type of nano-imprints exerted minimum effects on stem cells chondrogenesis as compared to that grown on a non-imprinted surface under the same chondrogenic conditions.

More advantageously, the provision of nano-imprints induces the attached stem cells to chondrogenesis even in two-dimensional condition. This is surprising because stem cells, in particular, mesenchymal stem cells (MSCs), do not undergo chondrogenic differentiation in two-dimensional condition even with the provision of full chondrogenic media (such as dexamethasone and transforming growth factor beta), unless maintained in a format that is conducive for precartilage condensation, such as in a three-dimensional pellet culture system. Hence, the method may optionally exclude the step of culturing the population of stem cells on a three-dimensional pellet culture system.

According to a second aspect, there is provided a cartilage graft comprising a population of stem cells differentiated chondrocytes disposed on a plurality of imprints on a substrate, said imprints being arranged in an ordered manner and configured to selectively promote chondrogenesis of the stem cells.

Advantageously, the imprints can be arranged in an ordered manner, in which the imprints are spaced apart from each other according to a defined value, on the cartilage graft, such that it is now possible to predict and clearly define the extent of stem cell chondrogenesis that would occur on the cartilage graft. Hence, this ensures that the extent of stem cell chondrogenesis can be selected and reproducible, leading to greater certainty when fabricating a cartilage graft to have a desired degree of stem cell chondrogenesis.

### Definitions

The following words and terms used herein shall have the meaning indicated:

The expression “promoting stem cell chondrogenesis” as used herein, is to be interpreted broadly to refer to an increase in the differentiation of stem cells, such as mesenchymal stem cells, into chondrocytes. This enables increased cartilage formation, enhanced production of cartilage matrix and/or increased cartilage repair.

The term “stem cells” is to be interpreted broadly to refer to undifferentiated cells that have the potential to differentiate into different cell types. The stem cells are able to self-renew in the sense that they can migrate to areas of injury to generate daughter cells that can undergo terminal differentiation into more than one distinct cell phenotype. As such, stem cells can enhance the regeneration or repair of a diseased or damaged tissue of interest. The stem cell may be a pluripotent stem cell or a multipotent stem cell.

The term “mesenchymal stem cell” refers to a multipotent stem cell that can differentiate into osteoblasts, chondrocytes, myocytes and adipocytes. When referring to bone or cartilage, MSCs are commonly known as osteochondrogenic, osteogenic, chondrogenic, or osteoprogenitor cells, since a single MSC has shown the ability to differentiate into chondrocytes or osteoblasts, depending on the medium.

The term “chondrocytes” refers to cells that are found in cartilage. Chondrocytes produce and maintain the cartilaginous matrix, which consists mainly of collagen and proteoglycans. From least to terminally differentiated, the chondrocytic lineage is (i) Colony-forming unit-fibroblast (CFU-F); (ii) mesenchymal stem cell/marrow stromal cell (MSC); (iii) chondrocyte; and (iv) hypertrophic chondrocyte. The term “chondrogenesis” refers to the formation of new cartilage from cartilage forming or chondrocompetent cells.

The term “biocompatible material” is to be interpreted broadly to refer to a material that would not induce an immune response (such as excessive fibrosis or rejection reactions) if transplanted or introduced into a biological system.

The term “selectively” refers to the ability to select or choose the extent or degree of stem cell chondrogenesis by adjusting the topography or imprints that the stem cells are grown on. The extent or degree of stem cell chondrogenesis may be one of high chondrogenic potential or low chondrogenic potential, when compared to identical stem cells grown on a substrate not having any topography or imprints thereon. The term “nanoimprinting lithography” is to be interpreted broadly to include any method for printing or creating a pattern or structure on the micro/nanoscale on the surface of a substrate by applying a mold with a defined imprint pattern or structure on the surface at certain temperature and pressure. A method of nanoimprinting lithography can be referred from U.S. Pat. No. 5,772,905.

The term “microscale” is to be interpreted to include any dimensions that are in the range of about 1 (μm) to about 100 μm. The term “microstructures” as used herein, refers to imprint structures comprising “microscale” features.

The term “nanoscale” is to be interpreted to include any dimensions that are below about 1 μm. The term “nanostructures” as used herein, are imprint structures comprising “nanoscale” or “submicron” features.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### Detailed Disclosure of Embodiments

Exemplary, non-limiting embodiments of a method for promoting stem cell chondrogenesis will now be disclosed.

The method for promoting stem cell chondrogenesis comprises the step of culturing a population of stem cells on a plurality of imprints disposed on a substrate, the imprints being configured to selectively promote chondrogenesis of the stem cells. As such, the disclosed method provides a means to select the degree of chondrogenesis of the stem cells by choosing the type of imprints that will result in the desired degree of chondrogenesis. Hence, this may allow greater certainty and reproducibility when growing the stem cells on a scaffold or support to form an artificial tissue in vitro.

The imprints may be arranged in an ordered manner on the substrate. The imprints may be arranged in an ordered array on the substrate such that the distance between adjacent imprints is of a set value. This distance may be obtained by measuring the distance between the center of one imprint and the center of another imprint that is adjacent to the first imprint. The center-to-center distance between adjacent imprints may be the same as the center-to-center distance of another pair of adjacent imprints. By having the imprints in an ordered manner (that is, not in a random configuration), the imprints can be reproducible such that the extent or degree of stem cell chondrogenesis on the imprints can occur in a predictive manner.

The imprints may be in the micro-scale or may be in the nano-scale. The imprints may be in the micro-scale in which at least one dimension of the imprint (height, length, width, breadth or diameter) may be in the micro-scale (herein after termed as “micro-imprint”). The micro-imprint may be selected from the group consisting of micro-pillar, micro-hole, micro-grill, micro-wire and micro-tube. The imprints may be in the nano-scale in which at least one dimension of the imprint (height, length, width, breadth or diameter) may be in the nano-scale (herein after termed as “nano-imprint”). The nano-imprint may be selected from the group consisting of nano-pillar, nano-hole, nano-grill, nano-wire and nano-tube. In general, stem cells grown on nano-imprints show a higher chondrogenic potential than stem cells grown in micro-imprints.

Where the nano-imprint is a nano-pillar, the diameter of the nano-pillar may be in the range of about 10 nm to about 500 nm, about 10 nm to about 300 nm, about 200 nm to about 300 nm and about 200 nm to about 500 nm. In one embodiment, the diameter of the nano-pillar may be about 250 nm. The pitch of the nano-pillar may be in the range of about 100 nm to about 1000 nm, about 100 nm to about 500 nm, about 500 nm to about 1000 nm and about 400 nm to about 600 nm. In one embodiment, the pitch of the nanopillar may be about 500 nm. The height of the nanopillar may be in the range of about 10 nm to about 500 nm, about 10 nm to about 300 nm, about 200 nm to about 300 nm and about 200 nm to about 500 nm. In one embodiment, the height of the nano-pillar may be about 250 nm. It is to be appreciated that the above ranges are not particularly limited and can be adjusted as desired.

Where the nano-imprint is a nano-hole, the diameter of the nano-hole may be in the range of about 10 nm to about 500 nm, about 10 nm to about 300 nm, about 200 nm to about 300 nm and about 200 nm to about 500 nm. In one embodiment, the diameter of the nano-hole may be about 225 nm. The pitch of the nano-hole may be in the range of about 10 nm to about 500 nm, about 10 nm to about 300 nm, about 300 nm to about 400 nm and about 300 nm to about 500 nm. In one embodiment, the diameter of the nano-hole may be about 300 nm. The height of the nano-hole may be in the range of about 10 nm to about 500 nm, about 10 nm to about 300 nm, about 300 nm to about 400 nm and about 300 nm to about 500 nm. In one embodiment, the height of the nano-hole may be about 300 nm. It is to be appreciated that the above ranges are not particularly limited and can be adjusted as desired.

The nano-imprint may be a nano-grill having a longitudinal axis that extends along the length of the nano-grill and being perpendicular to the width of the nano-grill, the longitudinal axis being parallel to the surface of the substrate. The width of the nano-grill may be in the range of about 10 nm to about 500 nm, about 10 nm to about 300 nm, about 200 nm to about 300 nm and about 200 nm to about 500 nm. In one embodiment, the width of the nano-grill may be about 250 nm. The spacing between adjacent nano-grills (or the center-to-center distance) may be in the range of about 10 nm to about 500 nm, about 10 nm to about 300 nm, about 200 nm to about 300 nm and about 200 nm to about 500 nm. In one embodiment, the spacing of the nano-grill may be about 250 nm. The height of the nano-grill (as measured from the bottom of the nano-grill to the top of the nano-grill) may be in the range of about 10 nm to about 500 nm, about 10 nm to about 200 nm, about 100 nm to about 500 nm and about 100 nm to about 200 nm. In one embodiment, the height of the nano-grill may be about 150 nm. It is to be appreciated that the above ranges are not particularly limited and can be adjusted as desired.

The method may comprise the step of forming the imprints on the substrate. The forming step is not particularly limited and may include any technique that is capable of forming an ordered set of imprints on a substrate. Exemplary techniques to form the imprints on the substrate may include thermal nanoimprinting or nanoimprint lithography.

In thermal imprinting or nanoimprint lithography, a mold having an imprint forming surface may be used. The mold may be made of a material that is able to withstand the elevated temperature used during imprinting and may be selected from the group consisting of silicon, metal, glass, quartz, ceramic or combinations thereof. The imprint forming surface of the mold may comprise imprints that are the inverse of the imprints that are to be formed on the substrate. The mold may be pressed onto a substrate at a defined temperature, pressure and/or time in order to form the imprints on the substrate. Typically, an elevated temperature is used to at least partially melt the substrate, forming a molten melt that can flow and conform to the shape of the mold. This may aid in ensuring that the imprints are formed on the substrate. In one embodiment, an imprinting temperature 80° C., an imprinting pressure of 60 bars, and an imprinting duration of 5 minutes may be used. The mold may be held together with the substrate for a sufficient period of time to ensure that the imprints are formed on the substrate before demolding the mold from the substrate. When demolding the mold from the substrate, the temperature is typically reduced in order to harden the substrate. In one embodiment, the demolding temperature may be room temperature. The mold and/or substrate may be treated with an anti-stiction agent before imprinting in order to aid the demolding of the mold from the substrate.

The method may comprise the step of coating the imprints with a chondrogenic inducing agent. The chondrogenic inducing agent may be selected from the group consisting of chondroitin sulphate, serum-free DMEM, ascorbate, dexamethasone, L-proline, sodium pyruvate, antibiotics and recombinant human transforming growth factor β1. The chondrogenic inducing agent may be immobilized as a layer onto the substrate. The chondrogenic inducing agent may be immobilized onto the substrate by chemical bonding or conjugation with appropriate chemical functional groups on the substrate surface. For example, if the chondrogenic inducing agent is chondroitin sulphate, the substrate surface may be treated with an amino solution in order to form an aminolyzed surface. The aminolyzed surface may be then treated with a carbodiimide solution and chondroitin sulphate. The carbodiimide solution functions to activate the carboxyl acid groups present on the chondroitin sulphate so as to allow the ‘activated’ chondroitin sulphate to covalently bind with the amino functional groups on the substrate surface in order to form a layer thereon the substrate.

The method may comprise the step of choosing the substrate from a biocompatible polymer. The biocompatible polymer may be selected from the group consisting of carbomer, polyalkylene glycol, poloxamer, polyester, polyether, polyanhydride, polyacrylate, polyvinyl acetate, polycarbonate, polyvinyl pyrrolidone, poly carboxylic acid, poly hydroxyacid, polygalpolysaccharide, polycarbonate, mixtures and copolymers of the polymers and monomers thereof. An exemplary biocompatible polymer may be selected from the group consisting of polycaprolactone, polymethyl methacrylate, polycarbonate, polylactic acid and poly(lactic-co-glycolic acid).

The method may comprise the step of selecting the stem cells from the group consisting of mesenchymal stem cells, neural stem cells, haemopoietic stem cells, endothelial stem cells and adipose-derived stem cells.

The method may comprise the step of selecting the type of imprints according to the desired degree of stem cell chondrogenesis. When the imprints are nano-pillars or nano-holes, the stem cells may selectively differentiate into cells with high chondrogenic potential, when compared to stem cells cultured on a substrate not having any imprints thereon. When the imprints are nano-grills, the stem cells may selectively differentiate into cells with low chondrogenic potential, when compared to stem cells cultured on a substrate not having any imprints thereon. The different types of differentiated cells as derived from the various topographical surfaces can be applied to different cartilage type. For example, cells with a higher chondrogenic potential can be applied to hyaline cartilage while cells with lower chondrogenic potential can be applied to fibrocartilage (which do not require collagen type II formation).

The stem cells differentiated chondrocytes may be provided on a scaffold to form a cartilage graft.

The cartilage graft comprises a population of stem cells differentiated chondrocytes disposed on a plurality of imprints on a substrate (forming the scaffold), the imprints being arranged in an ordered manner and configured to selectively promote chondrogenesis of the stem cells.

As mentioned above, the imprints may be arranged in an ordered manner on the substrate. The imprints may be in the micro-scale or may be in the nano-scale. The imprint may be a micro-imprint selected from the group consisting of micro-pillar, micro-hole, micro-grill, micro-wire and micro-tube. The imprint may be a nano-imprint selected from the group consisting of nano-pillar, nano-hole, nano-grill, nano-wire and nano-tube. The imprints may be formed on the substrate by thermal imprinting or nanoimprint lithography as mentioned above.

The substrate may be a biocompatible polymer selected from the group consisting of carbomer, polyalkylene glycol, poloxamer, polyester, polyether, polyanhydride, polyacrylate, polyvinyl acetate, polycarbonate, polyvinyl pyrrolidone, poly carboxylic acid, poly hydroxyacid, polygalpolysaccharide, polycarbonate, mixtures and copolymers of the polymers and monomers thereof. An exemplary biocompatible polymer may be selected from the group consisting of polycaprolactone, polymethyl methacrylate, polycarbonate, polylactic acid and poly(lactic-co-glycolic acid).

The substrate may be coated with a chondrogenic inducing agent selected from the group consisting of chondroitin sulphate, serum-free DMEM, ascorbate, dexamethasone, L-proline, sodium pyruvate, antibiotics and recombinant human transforming growth factor β1. The substrate may be coated with the chondrogenic inducing agent as mentioned above.

The population of stem cells differentiated chondrocytes may be derived from stem cells selected from the group consisting of mesenchymal stem cells, neural stem cells, haemopoietic stem cells, endothelial stem cells and adipose-derived stem cells, and mixtures thereof.

The stem cells differentiated chondrocytes on the cartilage graft may have a morphology selected from the group consisting of round morphology, polygonal morphology and spindle morphology.

The cartilage graft may provide a surface for cartilaginous tissue formation and/or other tissue formation such as bone growth or repair. The cartilage graft may be implantable in a patient in need thereof. The cartilage graft may be a component of a prosthetic limb. The cartilage graft may be a bone graft.

The cartilage graft may comprise, in addition to the population of stem cell differentiated chondrocytes, genetically engineered cells that are able to express biological agents such as proteins, growth factors, extracellular matrix materials (such as collagen or fibronectin) or other chondrogenesis stimulating agents that are necessary to form the cartilage graft. These biological agents may function to guide cell growth, synthesize extra cellular matrix or other biological molecules, and facilitate formation of functional tissues.

The cartilage graft may be a two-dimensional nano-topography scaffold. The cartilage graft may be constructed into a three-dimensional scaffold, which may lead to higher stem cell chondrogenesis.

The cartilage graft may be used to treat a patient suffering from a disease or disorder selected from the group consisting of osteochondritis dissecans, traumatic chondral fracture of the knee, osteoarthritis, achondroplasia, costochondritis, relapsing polychondritis, chondroma and chondrosarcoma. The cartilage graft may also be used to correct congenital ears and nose defects in children or used in rhinoplasty surgery.

There is also provided a method of treating a patient having a disease or disorder selected from the group consisting of osteochondritis dissecans, traumatic chondral fracture of the knee, osteoarthritis, achondroplasia, costochondritis, relapsing polychondritis, chondroma, chondrosarcoma, ear defect and nose defect, comprising the step of administering the cartilage graft to the patient.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a process diagram of patterning a substrate with nano-topographical features via thermal nanoimprinting.

FIG. 2 is a process diagram of the chemical conjugation of chondroitin Sulfate (CS) on nano-patterned substrate films.

FIG. 3a is a scanning electron microscopy (SEM) image of the surface of a polycaprolactone (PCL) substrate, patterned with nanopillars. The inset is a magnified image of the same. The scale bar of FIG. 3a is 1 μm while that of the inset is 200 nm.

FIG. 3b is a SEM image of the surface of a PCL substrate, patterned with nanoholes. The inset is a magnified image of the same. The scale bar of FIG. 3b is 1 μm while that of the inset is 200 nm.

FIG. 3c is a SEM image of the surface of a PCL substrate, patterned with nanogrills. The inset is a magnified image of the same. The scale bar of FIG. 3c is 1 μm while that of the inset is 200 nm.

FIG. 3d is a SEM image of the surface of a non-patterned PCL substrate. The scale bar of FIG. 3d is 1 μm.

FIG. 3e is a SEM image of the patterned PCL surface of FIG. 3a, coated with CS. The scale bar of FIG. 3e is 1 μm.

FIG. 3f is a SEM image of the patterned PCL surface of FIG. 3b, coated with CS. The scale bar of FIG. 3f is 1 μm.

FIG. 3g is a SEM image of the patterned PCL surface of FIG. 3c, coated with CS. The scale bar of FIG. 3g is 1 μm.

FIG. 3h is a SEM image of the non-patterned PCL surface of FIG. 3d, coated with CS. The scale bar of FIG. 3h 1 μm.

FIG. 4 is a graph showing the quantitative amount of CS conjugated on the non-patterned PCL films at different CS concentration over a period of 24 hours.

FIG. 5 is a graph showing the various cell proliferation profiles on the different nano-patterned CS-coated PCL films in comparison with the non-patterned CS-coated PCL film at different culture times over a period of 28 days.

FIG. 6a shows the cell morphology of chondrogenic induced mesenchymal stem cells (MSCs) on the CS-coated nanopillar PCL film after 48 hours in a chondrogenic culture. The scale bar of FIG. 6a is 5 μm.

FIG. 6b shows the cell morphology of chondrogenic induced MSCs on the CS-coated nano-hole PCL film after 48 hours in a chondrogenic culture. The scale bar of FIG. 6b is 5 μm.

FIG. 6c shows the cell morphology of chondrogenic induced MSCs on the CS-coated nano-grill PCL film after 48 hours in a chondrogenic culture. The scale bar of FIG. 6c is 5 μm.

FIG. 6d shows the cell morphology of chondrogenic induced MSCs on the CS-coated non-patterned PCL film after 48 hours in a chondrogenic culture. The scale bar of FIG. 6d is 5 μm.

FIG. 7a is a pictorial representation of the AFM cantilever used to carry out the indentation on the center of the cell using a 4.5 μm diameter bead attached to the AFM cantilever.

FIG. 7b is a pictorial representation of the deflection in the cantilever arm due to the indentation carried out on the centre of a cell using the 4.5 μm diameter bead attached to the AFM cantilever.

FIG. 8 is a bar graph of the various cell elasticities of the MSCs obtained from the various CS coated PCL films after 1 day and 3 days of culture, with P(x)=0.0172; P(*)=0.0381; P(**)=0.0134.

FIG. 9a is the rhodamine phalloidin-stained F-actin and anti-β-integrin-stained fluorescent image showing the F-actin reorganization and β1-Integrin expression of MSC on the nano-pillar PCL film after 3 days of culture under chondrogenic conditions. The scale bar of FIG. 9a is 1 μm.

FIG. 9b is an immunochemistry image showing the F-actin reorganization and β1-Integrin expression of MSC on the nano-hole PCL film after 3 days of culture under chondrogenic conditions. The scale bar of FIG. 9b is 1 μm.

FIG. 9c is an immunochemistry image showing the F-actin reorganization and β1-Integrin expression of MSC on the nano-grill PCL film after 3 days of culture under chondrogenic conditions. The scale bar of FIG. 9c is 1 μm.

FIG. 9d is an immunochemistry image showing the F-actin reorganization and β1-Integrin expression of MSC on the non-patterned PCL film after 3 days of culture under chondrogenic conditions. The scale bar of FIG. 9d is 1 μm.

FIG. 10a is an immunochemistry image showing the collagen type II expression of the MSCs on nano-pillar PCL film surface.

FIG. 10b is an immunochemistry image showing the collagen type II expression on nano-hole PCL film surface.

FIG. 10c is an immunochemistry image showing the collagen type II expression on nano-grill PCL film surface.

FIG. 10d is an immunochemistry image showing the collagen type II expression on non-patterned PCL film surface.

FIG. 10e is a Western blot showing the qualification of collagen type II expression of the MSCs on the different PCL films at week 4.

FIG. 10f is a bar graph showing the quantification of collagen type II expression of the MSCs by ELISA at week 4 and week 6.

FIG. 11a is a bar graph showing the quantification of the collagen type II markers expressed by the MSCs under chondrogenic conditions via the qRT-PCR method at week 2 and week 4 on the various types of PCL film surfaces.

FIG. 11b is a bar graph showing the quantification of the aggrecan markers expressed by the MSCs under chondrogenic conditions via the qRT-PCR method at week 2 and week 4 on the various types of PCL film surfaces.

FIG. 11c is a bar graph showing the quantification of the Sox9 markers expressed by the MSCs under chondrogenic conditions via the qRT-PCR method at week 2 and week 4 on the various types of PCL film surfaces.

FIG. 11d is a bar graph showing the quantification of the collagen X markers expressed by the MSCs under chondrogenic conditions via the qRT-PCR method at week 2 and week 4 on the various types of PCL film surfaces.

### DETAILED DESCRIPTION OF DRAWINGS

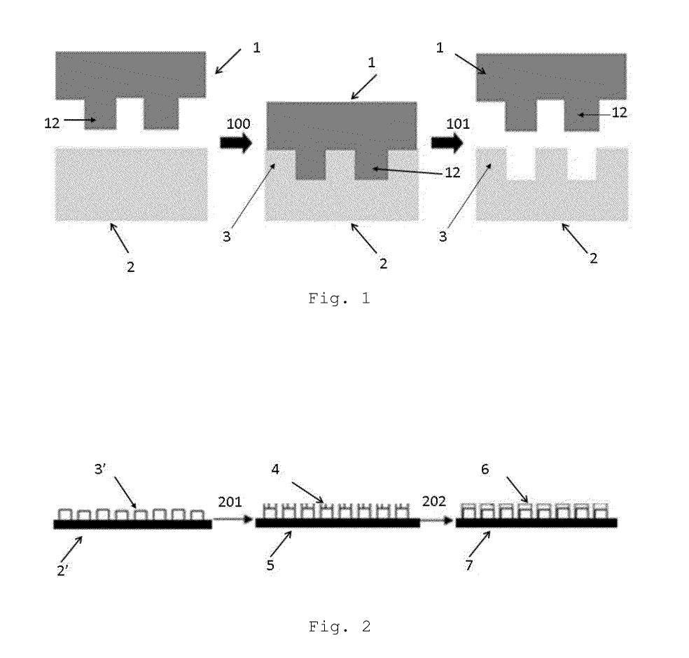

FIG. 1 is a process diagram showing the patterning of a substrate surface 2 with nanoimprints 3. A mold 1 with nanoimprints 12 that are the inverse of a desired nanoimprint 3 is prepared for contact with the substrate surface 2. The mold 1 was initially silanized with an anti-stiction monolayer and then thermally imprinted onto the substrate surface 2 at an elevated temperature and pressure (step 100). After a period of time, the temperature was reduced in order to allow demolding of the mold 1 from the substrate surface 2 (step 101). After the demolding step 101, nanoimprints 3 were formed on the substrate surface 2.

FIG. 2 is a process diagram showing the chemical conjugation of CS on the nano-patterned PCL film. Here, like reference numerals that were presented in the above figure are repeated here but with the prime (′) symbol. The substrate surface 2′ having the nanoimprints 3′ was incubated in an amine solution for a period of time (step 201) and washed to form an aminolyzed substrate 5. The aminolyzed substrate 5 with activated amino groups 4 was treated with a carbodiimide solution and CS solution at a period of time and temperature and then washed in step 202 to obtain the CS layer 6, resulting in the CS immobilized substrate 7.

### EXAMPLES

Non-limiting examples of the invention will be further described in greater detail by reference to specific Examples, which should not be construed as in any way limiting the scope of the invention.

In the examples, all assays were repeated with a minimum of 3 times. Data were analyzed using SPSS 10.0 software. Standard deviation was calculated by excel software. Statistically significant values were defined as p<0.05 based on one-way analysis of variance (ANOVA and T-test).

Cells were obtained from clinical patients. The chemicals were obtained as follows. Alginate, CaCl₂, EDTA, HA, chondroitin-6 sulfate, acid organe II, ascorbic acid, L-proline, dexamethasone, hemotoxylin, fast green, Safranin O and chitosan were obtained from Sigma-Aldrich (St Louis, Mo. of the United States of America). 2-(N-morpholine)-ethane sulphonic acid (MES), 3-dimethylaminopropyl-3-ethylcarbodiimide (EDC) and N-hydrosuccine hydrochloride (NHS) were obtained from Merck (Darmstadt, Germany). Hanks' balanced salt solution (HBSS), Dulbecco's minimum Eagle's medium (DMEM), fetal bovine serum (FBS), 0.05% trypsin were obtained from Invitrogen (Grand Island, N.Y. of the United States of America). Type II collagen (Col2) was obtained from Vitrogen (Cohesion Technologies, Palo Alto, Calif. of the United States of America) and ITS⁺premix was obtained from BD Bioscience Inc. (Franklin Lakes, N.J. of the United States of America). All other reagents used were of analytical grade.

### Example 1

### Preparation and Nanoimprinting of PCL Films

PCL films were prepared by using a thermo-pressure method. The starting material, PCL beads, were heat-pressed at 80° C., 500 bar for 12 hours in an argon environment, followed by cooling down to room temperature overnight.

The process of FIG. 1 was used to imprint a substrate 2 such as a PCL film to form the nanoimprints 3 on the PCL films. The mold 1 such as a silicon mold was commercially purchased, for example, the mold used to form nano-grills and nano-holes on the PCL films were obtained from Institute of Microelectronics (of Singapore) and the mold used to form nanopillars on the PCL film was obtained from NIL Technology. The various molds were fabricated by standard microelectronics processing. The molds were cleaned by sonication in acetone and isopropanol (IPA), followed by oxygen plasma (80W, 250 mTorr) for 3 minutes. Next, the molds were silanized with an anti-stiction monolayer (FDTS, (1H, 1H, 2H, 2H)-Perfluorodecyltrichlorosilane) using a Self-Assembled Monolayer Coater (AVC, Sorona) and rinsed with chloroform to get rid of physisorbed FDTS on the mold. Following which, the molds were rinsed with acetone and IPA, and blown dry. The silanization treatment was used to reduce the surface energy of the molds to facilitate easy de-molding of the mold from the PCL films. The silicon mold, having an imprint surface 12 that was the inverse of the desired nanoimprints 3 to be imprinted on the PCL films, was contacted with the PCL film via thermal nanoimprinting using an Obducat AB Nanoimprinter at an imprinting temperature of 80° C. and a pressure of 60 bars for 5 minutes. Following this, the temperature was cooled down to 25° C. and de-molding was performed at this temperature. The PCL film was thus patterned with the desired nanoimprints 3 from the silicon mold. Here, three types of nanoimprints were patterned on the respective PCL films, and are (a) nano-grill: 250 nm line, 250 nm space and 150 nm height; (b) nano-pillar: 250 nm diameter pillar, 500 nm pitch and 250 nm height; and (c) nano-hole: 225 nm diameter hole, 400 nm pitch and 300 nm height.

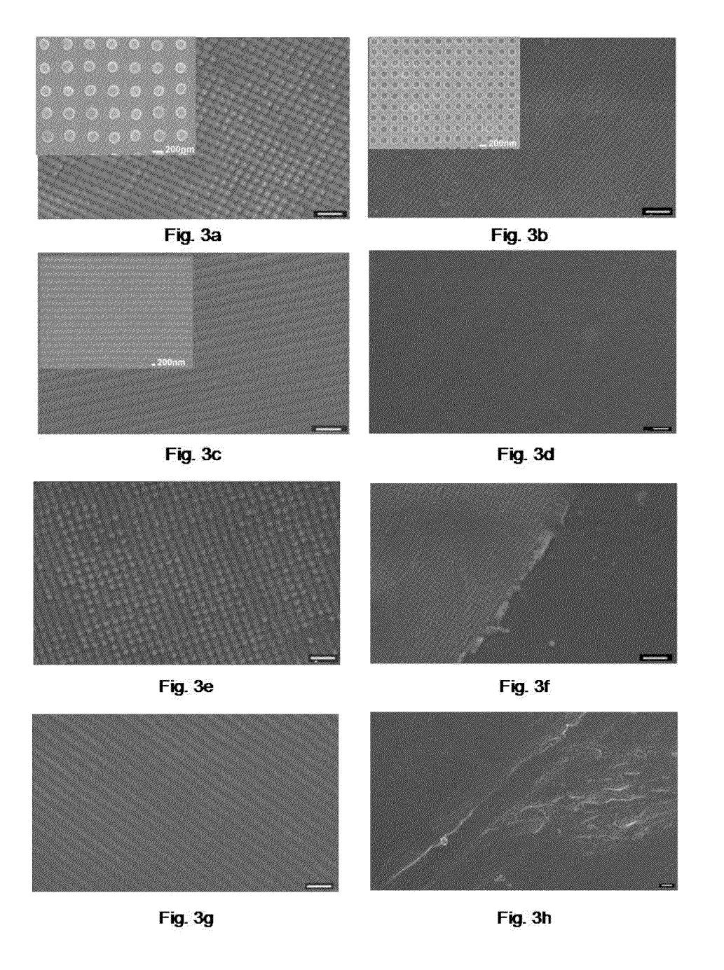

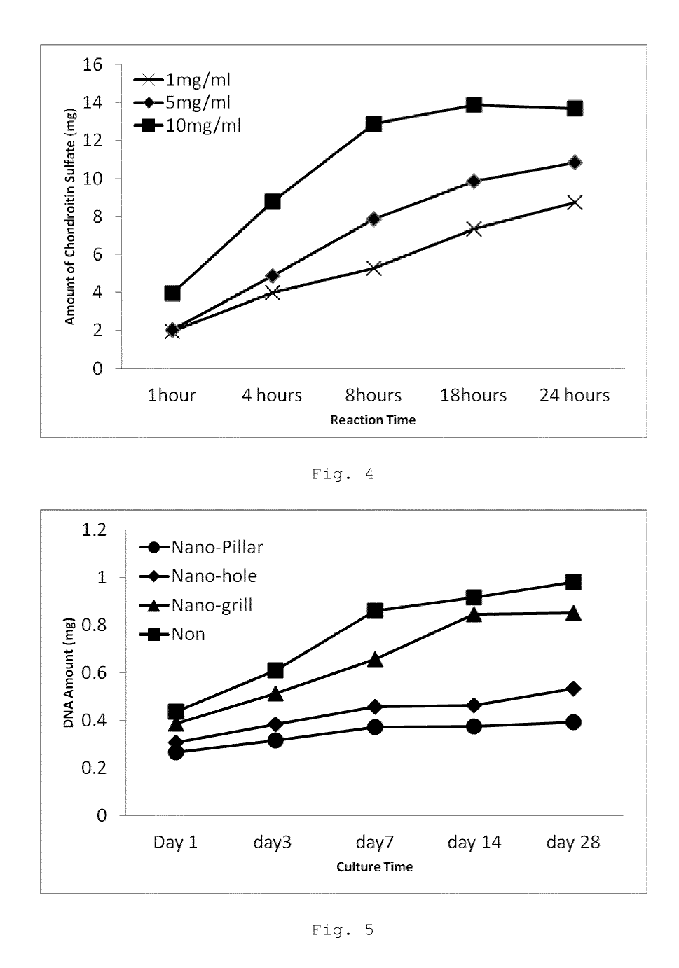

After thermal nanoimprinting and demolding, the morphology of the patterned PCL surfaces was examined by SEM. The morphologies of the pristine PCL film and the three nano-patterned PCL films were examined by using SEM (JEOL 5600). The surfaces of the PCL films were sputter-coated with about 20 nm gold film to prevent charging of the polymer film to facilitate better FESEM imaging. SEM image showed that the surface of the pristine PCL film was smooth (FIG. 3d). SEM images of the patterned PCL films showed that the surfaces were controllably patterned with the desired nanoimprints, namely:

nanopillars with 250 nm diameter and 500 nm pitch (FIG. 3a); nano-holes with 225 nm diameter and 400 nm pitch (FIG. 3b); and nano-grills of 250 nm line and 250 nm space (FIG. 3c). The nanoimprints were well-delineated and reliably replicated from the silicon mold.

### Example 2

### Chondroitin Sulfate (CS) Immobilization

FIG. 2 shows the process of chemical conjugation of CS to the substrate 2′ such as the PCL films. The various PCL films were incubated in 20% 1,6-Diaminohexane/ethanol solution for 15 minutes and the films were washed by PBS times for 30 minutes each time. The aminolyzed substrate 5 such as aminolyzed PCL films were treated with a carbodiimide solution (48 mM EDC and 6 mM NHS in 50 mM MES buffer, pH=5.5) and 10% CS MES solutions for 24 hours at 37.5° C. and then washed by PBS to obtain the CS layer 6 on the PCL films.

The surfaces of the CS-immobilized PCL films were imaged by SEM using the methodology described above. As can be seen in FIG. 3e to FIG. 3h, no significant morphological and dimensional changes were observed on the PCL films after they were subsequently coated with chondroitin sulfate.

The amounts of CS immobilized on the PCL films were measured based on the manufacturer's protocol (Blyscan™—Biocolor Life Science Assays). Briefly, the PCL films were immersed in 250 μl Blyscan dye and placed on a mechanical shaker for 2 hours. The films with Blyscan dye bound were washed by PBS 3 times. Unbound dye was removed completely and 600 μl dissociation reagent was used to cover the films for 1 hour to release bound dye. The absorbance of re-dissolved dye was measured in 96-well plates using FLUOstar Optima plate reader at absorbance wavelength of 656 nm. The standard curve of CS was generated from CS standard solution.

CS was used to provide the biochemical cue for MSC chondrogenesis on the PCL films. The amount of CS conjugated onto a nano-patterned PCL film was measured over a period of 24 hours. FIG. 4 shows that a higher concentration of CS solution resulted in a higher amount of CS conjugation onto the non-patterned PCL film. With 10 mg/ml CS solution, the amount of conjugated CS peaked (12.847 mg±0.826) after 8 hours reaction. On the other hand, the amount of conjugated CS using 5 mg/ml and 1 mg/ml CS solutions did not reach the maximum conjugation even after 24 hours. Thus, 10 mg/ml CS solution was used to coat the PCL film for subsequent experiments.

### Example 3

### Cell Proliferation of MSCs on PCL Films

The cell proliferations of MSCs on the various PCL films were calculated by the amount of DNA of MSCs on the various surfaces. The DNA amount was quantified fluorometrically using Hoechst Dye 33258 solution. Briefly, MSCs on the various PCL films were lysed in cell lysis buffer. Cell lysates were diluted 10 times and incubated with equal volume of 0.1 μg/ml Hoechest 33258 (Molecular Probes) solution for 10 minutes at room temperature, while protected from light, in 96-well black plates. Fluorescence was determined using a FLUOstar Optima fluorescent plate reader (BMG Labtech, Offenburg, Germany) at 350 nm excitation and 445 nm emission. The DNA concentrations of the samples, expressed as mg per liter, were extrapolated from standard curve generated using calf DNA.

The proliferation rates of MSCs cultured on the CS-coated nano-patterned PCL films were compared against the CS-coated non-patterned PCL film by DNA quantification over a period of 28 days (FIG. 5). MSCs on nano-grill and non-patterned surfaces showed the highest proliferation rate, yielding significantly higher DNA amount at day 14 compared to day 1 (P=0.024). MSCs proliferation on nano-grill surface was similar to that on non-patterned surface. Comparatively, MSCs on nano-pillar and nano-hole surfaces underwent negligible proliferation; the DNA amount throughout the culture period was not significantly higher than that at day 1 (P=0.201). The data implied that the proliferation profile of MSCs was affected by the different nanoimprints on the PCL films.

### Example 4

### Characterization of Chondrogenic Induced MSCs Morphology on PCL films and Surface Morphology of PCL Films

MSCs on PCL films were collected at 48 hours and fixed by 10% formalin 15 minutes. The samples were dehydrated sequentially in 50%, 75%, 95%, and 100% ethanol solutions. The dehydrated samples were air dried overnight and coated by gold for SEM imaging (FESEM) (JEOL 5600).

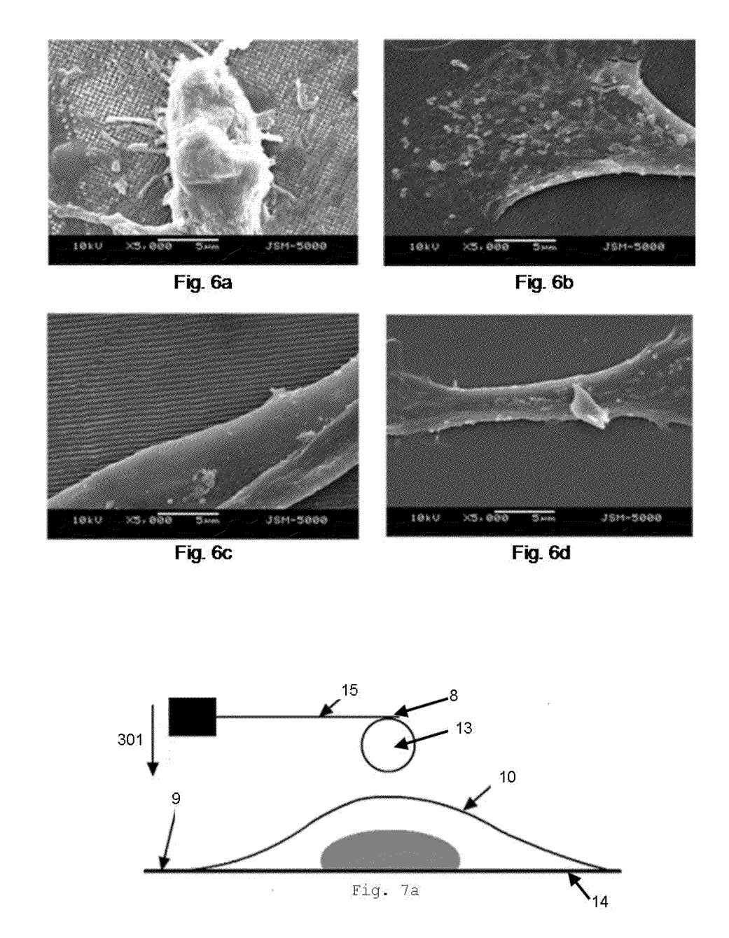

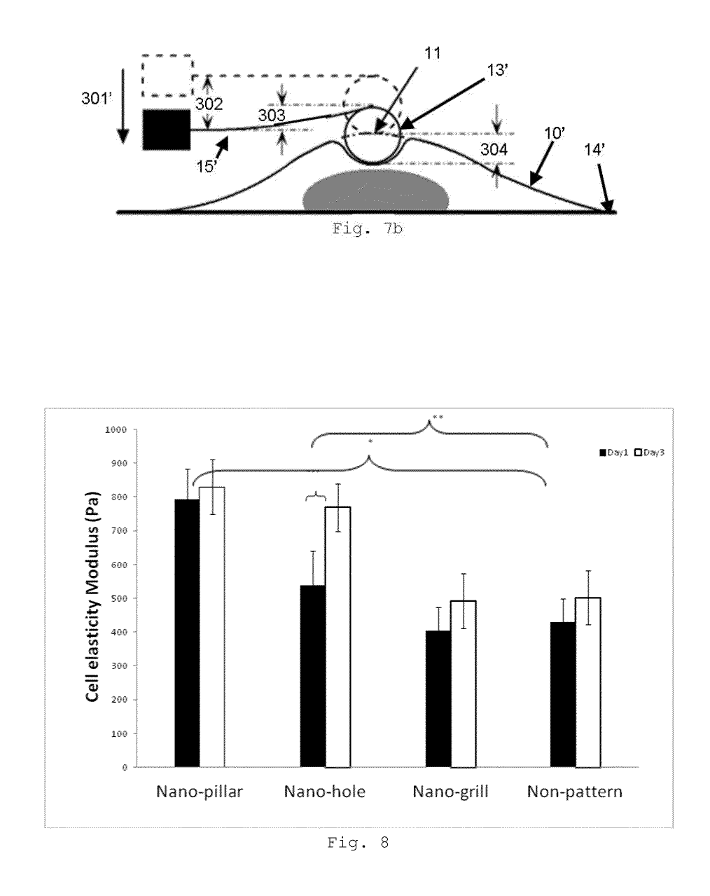

The SEM images showed significant differences in the morphologies of chondrogenic induced MSCs on different CS-coated PCL films after 48 hours of culture (FIGS. 6a to 6d). Induced MSCs on nano-pillar (FIG. 6a) surface showed round morphology with filipodial extrusion, while those on nano-hole (FIG. 6b) surface showed the polygonal morphology. The morphologies of induced MSCs on nano-grill (FIG. 6c) and non-patterned (FIG. 6d) surfaces adopted similar morphology, which were both spindle shape. The results indicated that cell morphologies of chondrogenic induced MSCs had been influenced by different nanoimprints on the PCL films.

In addition, SEM observations of the surface morphologies of the PCL films also showed some changes to the pattern dimensions. While nano-grill (FIG. 6c) and nano-pillar (FIG. 6a) PCL films did not show significant changes in their dimensions after 48 hours of incubation, the patterning of nano-hole (FIG. 6b) decreased after 48 hours culture. The nano-holes could not be observed in FIG. 6b when comparing between FIG. 6a to FIG. 6d using the same magnification. Qualitatively, this may indicate that the nano-holes had reduced in size since the nano-grill, nano-pillar and nano-hole were in the same dimensional range to begin with. Furthermore, the nano-holes should be observed given the same magnification but were not seen as expected.

### Example 5

### Measurement and Characterization of Cell Elasticity on the Chondrogenic Induced MSCs Cultured on PCL Films

A Nanoscope IV multimode AFM with a picoforce scanner (Digital Instruments Inc., USA) was used to carry out the experiments. FIG. 7a depicts the z-stage movement 301 of an AFM cantilever 15 used to carry out the indentation on the center of a cell using a 4.5 μm diameter bead 13 attached to the AFM cantilever 15. A modified silicon nitride AFM cantilever (NovaScan, USA) 15 having a spring constant of 0.01 N/m with a 4.5 μm diameter polystyrene bead tip 8 was used to indent the cells 10. During the experiment, a glass coverslip 9 grown with cells 10 was mounted on an AFM stage 14 and the cells 10 were kept in their culture medium using a standard fluid cell (Digital Instruments Inc., USA).

FIG. 7b depicts the z-stage movement distance 302 and the deflection in the cantilever arm 303 due to the indentation carried out on the centre of a cell using the 4.5 μm diameter bead 13′ attached to the AFM cantilever 15′ during stage movement 301′. Likewise, reference numerals that were presented in FIG. 7a are repeated here but with the prime (′) symbol. The relationship between the indentation depth 304 and the deflection of the cantilever 303 is also illustrated. Once the contact point 11 has been identified, the deflection-z position curve is converted to a plot of cantilever deflection against indentation depth 304, which can then be used to derive the apparent elastic modulus using the Hertz's model.

In this model, the apparent elastic modulus, E, can be obtained using the following formula:

$$H \cdot k = \frac{4}{3} \frac{E}{1 - {v}^{2}} \sqrt{{RD}^{3}}$$

where E is the apparent elastic modulus to be determined, v is the Poisson's ratio. R is the radius of the spherical bead, D is the indentation depth, H is the cantilever deflection and k is the spring constant of the cantilever. The cell was assumed incompressible and a Poisson's ratio of 0.5 was used.

The cell elasticities of chondrogenic induced MSCs cultured over 3 days on the various PCL films were compared (FIG. 8). On day 1, MSCs on the nano-pillar surface acquired significantly higher stiffness (793±90.39 Pa) compared to MSCs on non-patterned surface (327±60.27 Pa), while those on nano-grill surface remained similar (403±70.384 Pa) to that of non-patterned surface. Stiffness of MSCs on nano-hole surface increased significantly (P(x)=0.0172) from day 1 to day 3. On day 3, MSCs on nano-pillar surface maintained their stiffness (830 Pa±80.634 Pa) and stiffness of MSCs on nano-hole surface had increased to a level similar to those on nano-pillar surface (769 Pa±70.398). MSCs on both nano-grill and non-patterned surfaces remained low and unchanged from day 1.

### Example 6

### Immunochemistry Study of F-actin Reorganization and β-integrin Expression in Chondrogenic Induced MSCs on PCL Films

Chondrogenic induced MSCs on the various PCL films were collected at day 3 and fixed by 4% paraformaldehyde in PBS for 15 minutes at room temperature, followed by washing in PBS twice. The samples were incubated in 0.1% Triton X-100 in PBS for 5 minutes at room temperature and then washed in PBS twice. The samples were then blocked by 20% BSA in PBS for 30 minutes at room temperature and all samples were washed in PBS three times. The samples were incubated in anti-β-integrin antibody (diluted 1:1000) in 2% BSA/PBS solution for 2 hours at room temperature. The samples were washed by PBS three times (15 minutes each). A freshly prepared FITC conjugated second anti-body (goat anti-mouse) in 2% BSA/PBS solution was added onto the samples and incubated for 1 hour at room temperature. For double staining, the samples were incubated in TRITC-conjugated Phalloidin (diluted 1:500 in PBS) for 1 hour at room temperature after addition of the secondary antibody. The samples washed by PBS three times (10 minutes each). Finally, the DAPI/PBS (50 μg/ml) solutions were added onto the samples and incubated in the dark for 30 minutes at room temperature. The images were taken by Laser confocal microscopy (Olympus FV-1000) and processed by the software provided by Olympus.

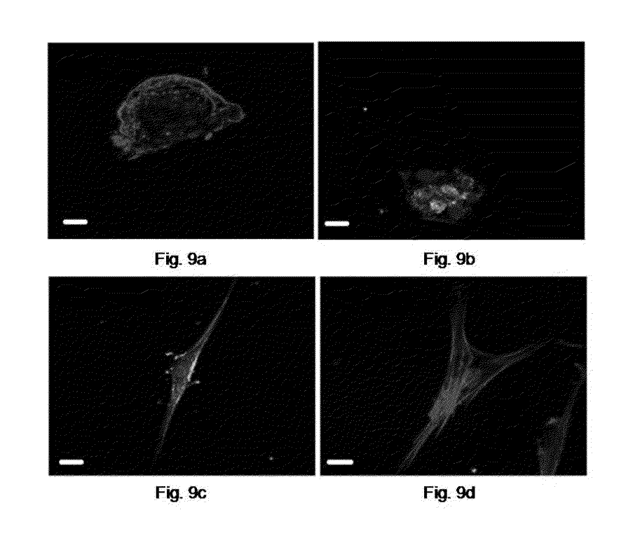

After 3 days of culture under chondrogenic conditions, the MSCs were fixed and immunofluorescent stained for F-actin and β1-integrin (FIGS. 9a to 9d). MSCs on nano-grill (FIG. 9c) and non-patterned (FIG. 9d) surfaces expressed fibrous F-actin and presented as spindle shape. MSCs on nano-pillar (FIG. 9a) and nano-hole (FIG. 9b) surfaces formed aggregates and F-actin organized at the cortical of the rounded cells surrounding the cell cluster. The F-actin expression of MSCs cluster on nano-pillar (FIG. 9a) surface was stronger than that on nano-hole (FIG. 9b) surface. The integrin expression was highest on the induced MSCs attached on nano-hole (FIG. 9b) surface. On the contrary, induced MSCs on nano-grill (FIG. 9c) and nano-pillar (FIG. 9a) surfaces expressed weak integrin expression, while almost no integrin expression was detected on MSCs attached on the non-patterned (FIG. 9d) surface.

### Example 7

### Analysis of Chondrogenic Differentiation of MSCs on PCL Films via Collagen Type II Analysis

The specific chondrogenic protein marker, collagen type II, had been analyzed by ELISA, Western blot and immunochemistry methods.

For the ELISA method, the collagen type II ELISA followed the manufacturer's protocol (Chondrex). Briefly, the samples were digested by pepsin in 0.05 M acetic acid for 4 days at 4° C. The supernatant was collected for the subsequently measurement. 10 μl collagen type II antibodies (1:100) were added to each well of the ELISA plate and incubated at 4° C. overnight. Following which, the ELISA plate was washed by wash buffer 6 times. 100 μl standard and samples were added to each well and kept in room temperature for 2 hours. The wells were washed by wash buffer 6 times. 100 μl detection antibody was added to each well and incubated at room temperature for 2 hours. The samples were washed again 6 times. 100 μl of the streptavidin peroxadise solution was added to each well and incubated at room temperature for 1 hour. One vial of OPD was dissolved in 10 ml dilution solution buffer, added to each well and incubated at room temperature for 30 minutes. The reaction was stopped by 50 μl 2N sulphuric acid and the ELISA plate was read at 490 nm. The collagen II amount was extrapolated from the collagen II standard curve.

For the Western Blot method, the samples were harvested and digested by pepsin/1.0N HCl for 1 hour. The solutions were concentrated by filter column (Millipore) and re-dissolved in 0.01N HCl. The samples were separated in SDS precast gel (Invitrogen) (120V and 90 minutes). The gel was collected according to the manufacturer's protocol and transferred to a nitrocellulose membrane (50V, Room temperature, overnight). The transferred membrane was washed by PBS three times and blocked by 20% milk for 2 hours at room temperature. The membrane was incubated in mouse anti-human collagen antibody (1:1000, in 2% milk) overnight at 4° C. The membrane was washed by PBS three times (10 minutes each) and incubated with the secondary antibody (goat anti-mouse HRP-antibody) for 1 hour at room temperature. Finally, the Western Blot development kit was used to create the chemi-luminescence and imaged by VanDoc machine.

For the immunochemistry method, the immunochemistry staining method was the same as the immunochemistry method used previously in the integrin staining method. The collagen type II antibody dilution ratio was 1:500.

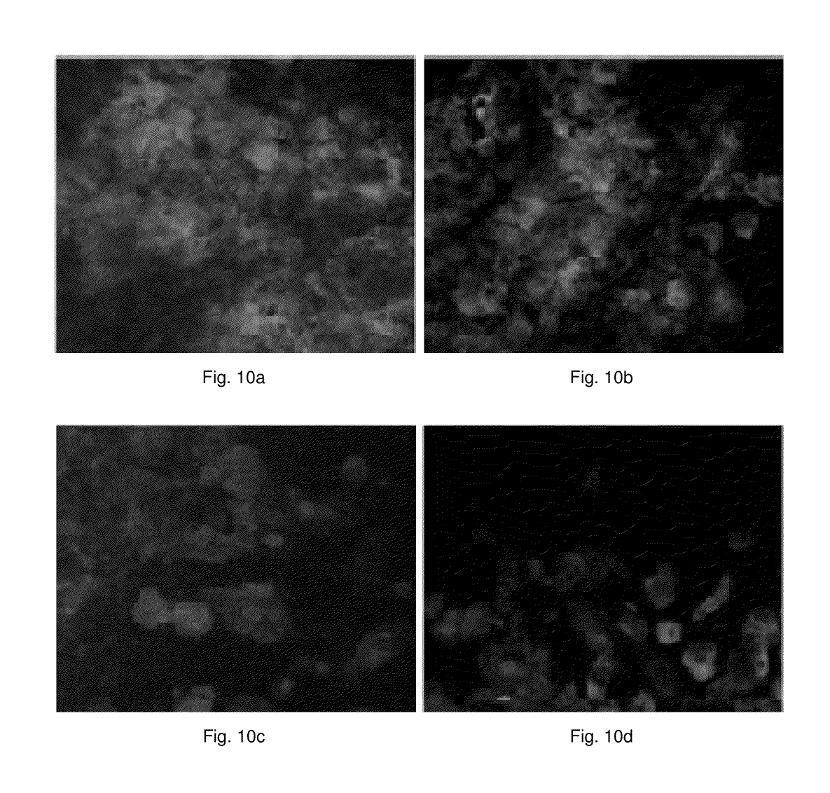

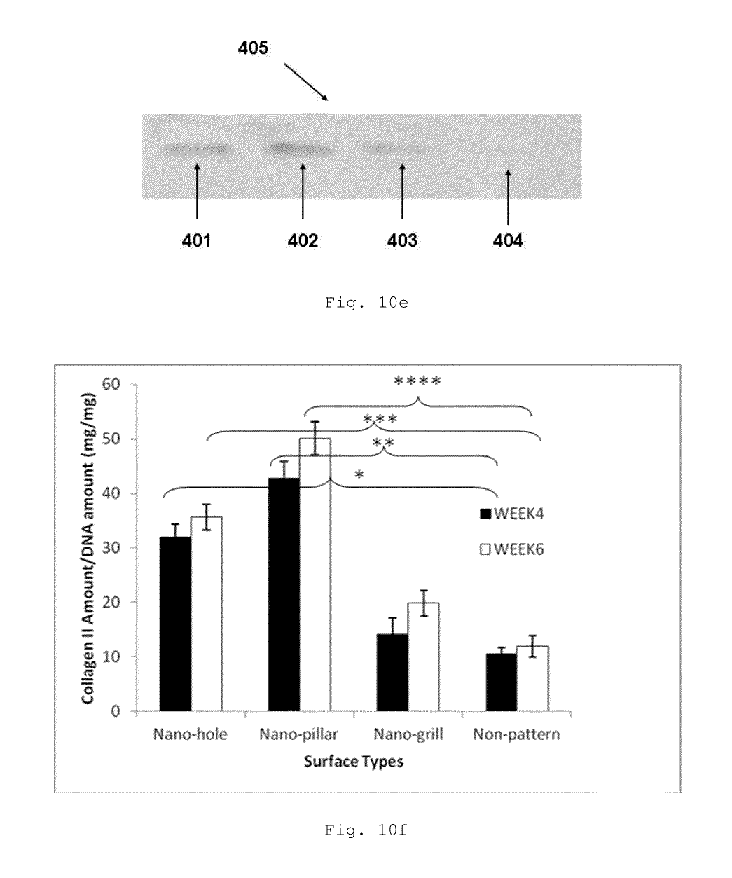

The degrees of chondrogenic differentiation of MSCs on the various PCL films were analysed by immunostaining and quantification of the expression level of the chondrogenic marker, collagen type II. In immunochemistry images (FIGS. 10a to 10d), MSCs on nano-pillar (FIG. 10a) surface shows the highest collagen type II expression. Higher collagen density also showed up in the nano-hole (FIG. 10b) and nano-pillar (FIG. 10a) surfaces compared to nano-grill (FIG. 10c) and non-patterned (FIG. 10d) surfaces, which only presented around the MSCs.

A similar trend was also indicated by the Western Blot analysis (see FIG. 10e). FIG. 10e shows the qualification of collagen type II expression on the various PCL films by Western Blot 405 at week 4. The qualification of collagen type II expression by Western Blot 405 for nano-pillar surface 402 was found to be highest. Nano-hole surface 401 also showed higher density of collagen expression compared to nano-grill 403 and non-patterned 404 surfaces.

Collagen type II level, measured by ELISA was normalized to the DNA amount (FIG. 10f). Consistent with the qualitative results, MSCs on nano-pillar surface expressed the highest amount of collagen at a level of 4 to 5 folds higher than that on the non-patterned surface. MSCs on nano-hole surface expressed collagen type II at a level of 3 folds higher, while those on nano-grill expressed similar level to that on non-patterned surface.

### Example 8

### Real Time PCR analysis for Quantification of Chondrogenic Related mRNA Markers

Cells were collected by trypsinization and total RNA was extracted using the RNeasy Mini Kit (Qiagen, Valencia, Calif.) following the manufacturer's protocol. RNA concentration was determined using the NanoDrop (NanoDrop Technologies, Wilmington, Del.) and reverse transcription reactions were performed with 50 ng total RNA using iScript™ cDNA synthesis kit (Biorad Laboratories, Hercules, Calif.). Real-time PCR reactions for GAPDH, Sox9, aggrecan, collagen II and collagen X were conducted using the SYBR green system. Real Time RCR reactions using the ABI 7500 Real Time PCR System (Applied Biosystems) were performed at 95° C. for 10 minutes, followed by 40 cycles of amplifications, which consisted of a denaturation step at 95° C. for 15 seconds, and an extension step at 60° C. for 1 minute. The level of expression of the target gene, normalized to GAPDH, was then calculated using the 2^{-ΔΔCt} formula with reference to the undifferentiated MSC.

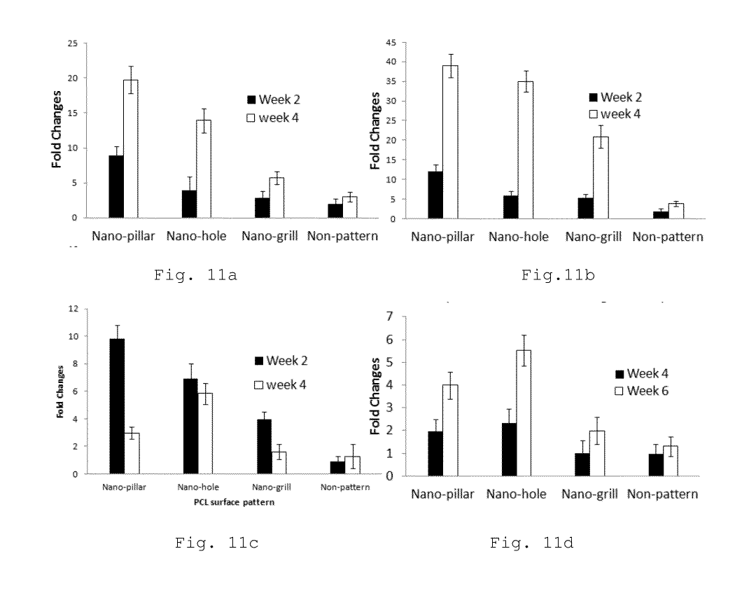

Real-time PCR analysis was used to quantify the chondrogenic related mRNA markers from induced MSCs on different nano-patterned surfaces versus the control non-patterned surface over week 2 and week 4 of cell culture under chondrogenic medium (FIGS. 11a to 11d). Sox9 (see FIG. 11c), which is one of the earliest markers expressed in cells undergoing chondrogenesis, was expressed at the highest levels on nano-pillar surface at week 2 but was overtaken by the nano-hole surface at week 4. Comparatively, Sox9 expressions on nano-grill and non-patterned surfaces were relative lower than the nano-pillar and nano-hole surfaces at both week 2 and week 4. Aggrecan and collagen II markers (see FIG. 11b and 11a respectively), which indicate chondrogenic matrix expression at gene levels, were compared. Induced MSCs showed highest aggrecan and collagen II mRNA synthesis on nano-pillar surface at both week 2 and week 4, followed by nano-hole surface. Non-patterned surface presented very low amounts of aggrecan and collagen II expression. Expression of aggrecan and collagen II on nano-grill surface was only up-regulated at the later time point of week 4, at levels 4- and 2-folds lower than that of nano-pillar. Collagen X mRNA (FIG. 11d), which represents hypertrophic cartilage tissue matrix expression was analyzed at week 4 and week 6 after differentiation. At week 4, induced MSCs on nano-pillar and nano-hole surfaces have up-regulated expression of collagen X mRNA, which persisted to week 6 in which significantly higher expression was detected with MSCs on the nano-hole surface than on the nano-pillar surface. MSCs on nano-grill and non-patterned surfaces exhibited no up-regulation of collagen X at all time points.

In conclusion, the above examples show that MSC chondrogenesis is sensitive to the type of nanoimprints or nano-topography on the growth substrate. The above results have shown how MSCs adopt specific morphology, undergone distinct cytoskeletal reorganization, change cellular membrane stiffness as it undergoes chondrogenesis at early stage and the onset of chondrogenesis on different surface topography. At early stage chondrogenesis, MSCs on nano-pillar and nano-hole topography adopted a spherical and polygonal morphology respectively, while MSCs on nano-grill and non-patterned topography adopted a spindle-shape morphology. Furthermore, aggregated cells on nano-pillar and nano-hole topography had their F-actin cytoskeleton reorganized into a cortical pattern, while aggregated cells on nano-grill and non-patterned topography presented fibrous F-actin. Cell membrane elasticity on nano-pillar and nano-hole topography became significantly stiffer compared to those on nano-grill and non-patterned topography. When these results were correlated with chondrogenic marker analysis, they provided valuable information and relationship between cell morphology, cytoskeletal reorganization and cellular membrane stiffness with chondrogenic ability. Spindle cell shape at early stage chondrogenesis predisposed cells to low chondrogenic ability whereas round and spread (polygonal) cell shapes induce a higher chondrogenic potential. At the onset of chondrogenesis, aggregated cells with their F-actin cytoskeleton reorganized into a cortical pattern and with stiffer cell membranes are likely to have enhanced chondrogenesis ability.

### Applications

The disclosed method may be used in tissue engineering to mitigate the unpredictability associated with stem cell chondrogenesis on random topographies. The disclosed method may be used to manufacture artificial tissues or cartilage that contain stem cell chondrocytes of a desired morphology and biological characteristics. The stem cells may be isolated from their original biological environment (such as bone marrow) and used as source cells for the differentiation of chondrocytes.

The disclosed method of nano-topography fabrication may allow a high degree of spatial control over the surface topography of the substrate or scaffold not present in other methods that produces random topography. The nano-topography fabricated scaffold may also enhance the studies of subsequent tissue responses to the topographical cues of the scaffold support. These nano-topographical features may further influences cell proliferation, cell adhesion, growth, migration, and other differentiated functions.

The disclosed method may allow the stem cells to differentiate in a two-dimensional environment due to the presence of the imprints on the substrate.

The disclosed method may not require complicated techniques such as phase separation, electrospinning or gel formation.

The disclosed cartilage graft may be used to solve issues associated with tissues or cells that have poor self-regenerative capacity due to low cellular mitotic activities, low supply of progenitor cells and their avascularization.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method for promoting stem cell chondrogenesis, comprising the step of culturing a population of stem cells on a plurality of imprints disposed on a substrate, said imprints being configured to selectively promote chondrogenesis of said stem cells.

2. The method according to claim 1, wherein said imprints are arranged in an ordered manner on said substrate.

3. The method according to claim 1, wherein said imprints are in the nano-scale.

4. The method according to claim 2, wherein said imprints are arranged in an ordered array on said substrate such that the distance between adjacent imprints is of a set value.

5. The method according to claim 1, wherein said imprints are formed on said substrate using thermal nanoimprinting or nanoimprint lithography.

6. The method according to claim 1, comprising the step of coating said imprints with a chondrogenic inducing agent.

7. The method according to claim 6, wherein said chondrogenic inducing agent is selected from the group consisting of chondroitin sulphate, serum-free DMEM, ascorbate, dexamethasone, L-proline, sodium pyruvate, antibiotics and recombinant human transforming growth factor β1.

8. The method according to claim 3, wherein said nano-imprint is selected from the group consisting of nano-pillar, nano-hole, nano-grill, nano-wire and nano-tube.

9. The method according to claim 1, wherein said substrate is a biocompatible polymer selected from the group consisting of carbomer, polyalkylene glycol, poloxamer, polyester, polyether, polyanhydride, polyacrylate, polyvinyl acetate, polycarbonate, polyvinyl pyrrolidone, poly carboxylic acid, poly hydroxyacid, polygalpolysaccharide, polycarbonate, mixtures and copolymers of the polymers and monomers thereof.

10. The method according to claim 9, wherein said biocompatible polymer is selected from the group consisting of polycaprolactone, polymethyl methacrylate, polycarbonate, polylactic acid and poly(lactic-co-glycolic acid).

11. The method according to claim 1, wherein said stem cells are selected from the group consisting of mesenchymal stem cells, neural stem cells, haemopoietic stem cells, endothelial stem cells and adipose-derived stem cells.

12. The method according to claim 8, wherein when nano-pillars or nano-holes are used, said stem cells selectively differentiate into cells with high chondrogenic potential and when nano-grills are used, said stem cells selectively differentiate into cells with low chondrogenic potential when compared to stem cells cultured on a substrate not having any imprints thereon.

13. A cartilage graft comprising a population of stem cells differentiated chondrocytes disposed on a plurality of imprints on a substrate, said imprints being arranged in an ordered manner and configured to selectively promote chondrogenesis of said stem cells.

14. The cartilage graft according to claim 13, wherein said imprint is a nano-imprint selected from the group consisting of nano-pillar, nano-hole, nano-grill, nano-wire and nano-tube.

15. The cartilage graft according to claim 13, said substrate is a biocompatible polymer selected from the group consisting of carbomer, polyalkylene glycol, poloxamer, polyester, polyether, polyanhydride, polyacrylate, polyvinyl acetate, polycarbonate, polyvinyl pyrrolidone, poly carboxylic acid, poly hydroxyacid, polygalpolysaccharide, polycarbonate, mixtures and copolymers of the polymers and monomers thereof.

16. The cartilage graft according to claim 13, wherein said stem cells are selected from the group consisting of mesenchymal stem cells, neural stem cells, haemopoietic stem cells, endothelial stem cells and adipose-derived stem cells.

17. The cartilage graft according to claim 13, wherein said chondrocytes have a morphology selected from the group consisting of round morphology, polygonal morphology and spindle morphology.

18. A method of treating a patient having a disease or disorder selected from the group consisting of osteochondritis dissecans, traumatic chondral fracture of the knee, osteoarthritis, achondroplasia, costochondritis, relapsing polychondritis, chondroma, chondrosarcoma, ear defect and nose defect, comprising the step of administering the cartilage graft according to claim 13 to said patient.
